(19) United States
(12) Patent Application Publication
(10) Pub. No.: US 2024/0268955 A1
(43) Pub. Date: Aug. 15, 2024

(54) Prosthetic Heart Valve Frame with Double-Arm Connection

(71) Applicant: Cephea Valve Technologies, Inc., Abbott Park, IL (US)

(72) Inventors: Zachary R. Vidlund, Robbinsdale, MN (US); Cathy Bergin, Hugo, MN (US)

(73) Assignee: Cephea Valve Technologies, Inc., Abbott Park, IL (US)

(21) Appl. No.: 18/428,380

(22) Filed: Jan. 31, 2024

Related U.S. Application Data
(60) Provisional application No. 63/484,017, filed on Feb. 9, 2023.

(51) Int. Cl. A61F 2/24

(52) U.S. Cl. CPC A61F 2/243; A61F 2/2418; A61F 2230/0017

(57) ABSTRACT

A prosthetic heart valve includes collapsible and expandable inner and outer frames. The outer frame engages native heart valve annulus tissue, with an atrial portion, a ventricle portion, and a narrowed waist portion between the two. The inner frame is positioned radially inward of the outer frame. The outer frame includes a plurality of outer coupling arms having a first end coupled to the outer frame and a second free end, and the inner frame includes a plurality of inner coupling arms having a first end coupled to the inner frame and a second free end. A prosthetic valve assembly is disposed within the inner frame. The first end of the inner and outer coupling arms each include two struts. The coupling arms extend toward each other and the second free ends of the coupling arms are coupled to each other to couple the outer frame to the inner frame.

### CROSS-REFERENCE TO RELATED APPLICATIONS

The present application claims priority to U.S. Provisional Ser. No. 63/484,017, filed Feb. 9, 2023, the disclosure of which is hereby incorporated by reference in its entirety as if fully set forth herein.

### BACKGROUND OF THE DISCLOSURE

Heart valve disease is a significant cause of morbidity and mortality. A primary treatment of this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after re-expansion.

### BRIEF SUMMARY

According to one aspect of the disclosure, a prosthetic heart valve includes a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end. A collapsible and expandable inner frame may be positioned radially inward of the outer frame, the inner frame including a plurality of inner coupling arms having a first end coupled to the inner frame and a second free end. A prosthetic valve assembly may be coupled to and disposed within the inner frame. The first ends of the outer coupling arms may each include at least two struts (including exactly two struts) extending radially inwardly from a remainder of the outer frame. The first ends of the inner coupling arms may each include at least two struts (including exactly two struts) extending radially outwardly from a remainder of the inner frame. The second free ends of the outer coupling arms may be coupled to the second free ends of the inner coupling arms to couple the outer frame to the inner frame. The first ends of the outer coupling arms may be coupled to the outer frame at a location substantially equidistant between an inflow end of the outer frame and an outflow end of the outer frame. The first ends of the inner coupling arms may be coupled to the inner frame at a location substantially equidistant between an inflow end of the inner frame and an outflow end of the inner frame. The outer coupling arms may be integral with the outer frame, and the inner coupling arms may be integral with the inner frame. The second free ends of the outer coupling arms may be coupled to the second free ends of the inner coupling arms via mechanical fasteners. The mechanical fasteners may be sutures. In an expanded condition of the outer frame, the outer coupling arms may be contoured so that the second free ends of the outer coupling arms are substantially parallel to a central longitudinal axis of the outer frame, the central longitudinal axis extending from an inflow end of the outer frame to an outflow end of the outer frame. In an expanded condition of the inner frame, the inner coupling arms may be contoured so that the second free ends of the inner coupling arms are substantially parallel to a central longitudinal axis of the inner frame, the central longitudinal axis extending from an inflow end of the inner frame to an outflow end of the inner frame.

The inner frame may include a plurality of rows of substantially diamond-shaped cells, including a first row at an inflow end of the inner frame. In a collapsed condition of the inner frame, each inner coupling arm may be nested within one of the cells in the first row of cells at the inflow end of the inner frame. The inner frame may include a plurality of axial struts, each axial strut extending from an outflow apex of a corresponding diamond-shaped cell within a row of diamond-shaped cells in a direction away from an inflow end of the inner frame, the axial struts defining commissure windows, the prosthetic leaflets of the prosthetic valve assembly being coupled to the axial struts via the commissure windows. Each axial strut may be coupled to two cells within the row of diamond-shaped cells via two support struts that each extends between the axial strut and a corresponding one of the two cells. Each support strut may have a first end coupled to a terminal end outflow end of the axial strut. The two struts of the outer coupling arm may converge to the second free end of the outer coupling arm, an aperture being formed in the second free end of the outer coupling arm. The two struts of the inner coupling arm converge to the second free end of the inner coupling arm, an aperture being formed in the second free end of the inner coupling arm. A suture may extend through the aperture in the second free end of the outer coupling arm and through the aperture in the second free end of the inner coupling arm to couple the outer coupling arm to the inner coupling arm.

The inner frame and the outer frame may each be formed from a nickel-titanium alloy. In an expanded condition of the prosthetic heart valve, the second free ends of the outer coupling arms may meet the second free ends of the inner coupling arms at a location that is substantially equidistance between the inner frame and the outer frame. Each inner coupling arm may have two separate points of connection to the inner frame so that the inner coupling arm is resistant to side-to-side bending, and each outer coupling arm may have two separate points of connection to the outer frame so that the outer coupling arm is resistant to side-to-side bending. Each inner coupling arm may have two separate points of connection to the inner frame so that the inner coupling arm is resistant to twisting upon application of torque, and each outer coupling arm may have two separate points of connection to the outer frame so that the outer coupling arm is resistant to twisting upon application of torque. Each inner coupling arm may have two separate points of connection to the inner frame i) so that the inner frame is resistant to axial travel relative to the outer frame, ii) to promote symmetric collapse and expression of the inner and outer frames, and/or iii) to reduce sheathing strains prior to and/or during delivery of the prosthetic heart valve. The inner frame may include a plurality of axial struts, each axial strut extending from a single outflow apex of a corresponding diamond-shaped cell within a row of diamond-shaped cells in a direction away from an inflow end of the inner frame, the axial struts defining partially-open commissure windows each defining a pair of freely extending tips, the prosthetic leaflets of the prosthetic valve assembly being coupled to the axial struts via the commissure windows.

### BRIEF DESCRIPTION OF THE DRAWINGS

FIG. 1A is a side view of an outer frame of a prosthetic heart valve in an expanded condition.

FIG. 1B is a flattened view of the outer frame of FIG. 1A, as if cut longitudinally and laid out flat on a table in an unexpanded condition.

FIGS. 1C-D are perspective and side views, respectively, of an inner frame of a prosthetic heart valve, which may be configured for use with the outer frame of FIGS. 1A-B.

FIG. 1E is a flattened view of the inner frame of FIGS. 1C-D, as if cut longitudinally and laid out flat on a table in an unexpanded condition.

FIG. 1F is a side view of a portion of an inner frame of a prosthetic heart valve, which may be configured for use with the outer frame FIGS. 1A-B.

FIGS. 2A-C are schematic illustrations showing a portion of the inner frame of FIGS. 1C-E coupled to different-sized outer frames.

FIGS. 3A-B are top and side views of a prosthetic heart valve, according to another aspect of the disclosure, with one-quarter of the valve cut away for purposes of illustration.

FIG. 3C is a perspective view of an outer frame of the prosthetic heart valve of FIGS. 3A-B in an expanded condition.

FIG. 3D is a perspective view of an inner frame of the prosthetic heart valve of FIGS. 3A-B in an expanded condition.

FIG. 4A is an isolated side view of a single-strut coupling arm of an inner frame, similar to that shown in FIGS. 1C-D.

FIG. 4B is an isolated side view of a double-strut coupling arm of an outer frame coupled to a double-strut coupling arm of an inner frame, similar to that shown in FIGS. 3C-D.

### DETAILED DESCRIPTION

As used herein, the term “inflow end,” when used in connection with a prosthetic heart valve, refers to an end of the prosthetic heart valve into which blood first flows when the prosthetic heart valve is implanted in an intended position and orientation. On the other hand, the term “outflow end,” when used in connection with a prosthetic heart valve, refers to the end of the prosthetic heart valve through which blood exits when the prosthetic heart valve is implanted in an intended position and orientation. In the figures, like numbers refer to like or identical parts. As used herein, the terms “substantially,” “generally,” “approximately,” and “about” are intended to mean that slight deviations from absolute are included within the scope of the term so modified. When ranges of values are described herein, those ranges are intended to include sub-ranges. For example, a recited range of 1 to 10 includes 2, 5, 7, and other single values, as well as all sub-ranges within the range, such as 2 to 6, 3 to 9, 4 to 5, and others.

The present disclosure is generally directed to collapsible prosthetic mitral valves, and in particular various features of stents thereof to provide enhanced functionality. However, it should be understood that the features described herein may apply to other types of prosthetic heart valves, including prosthetic heart valves that are adapted for use in other heart valves, such as the tricuspid heart valve. Further, the features of the prosthetic heart valves described herein may, in some circumstances, be suitable for surgical (e.g. non-collapsible) prosthetic heart valves. However, as noted above, the disclosure is provided herein in the context of a collapsible and expandable prosthetic mitral valve.

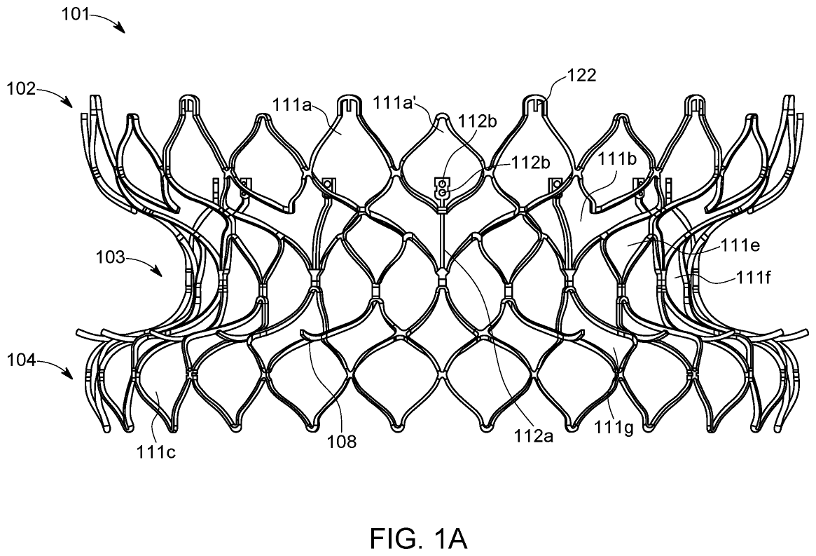

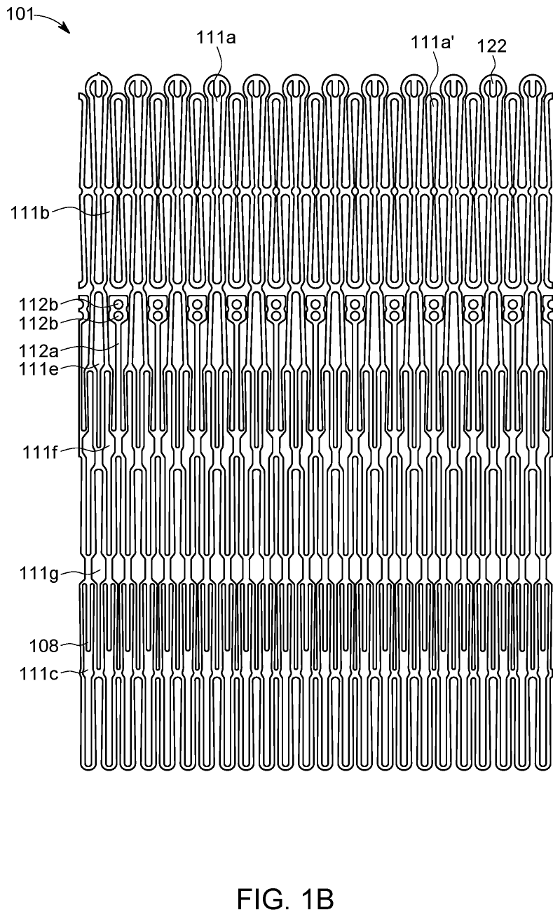

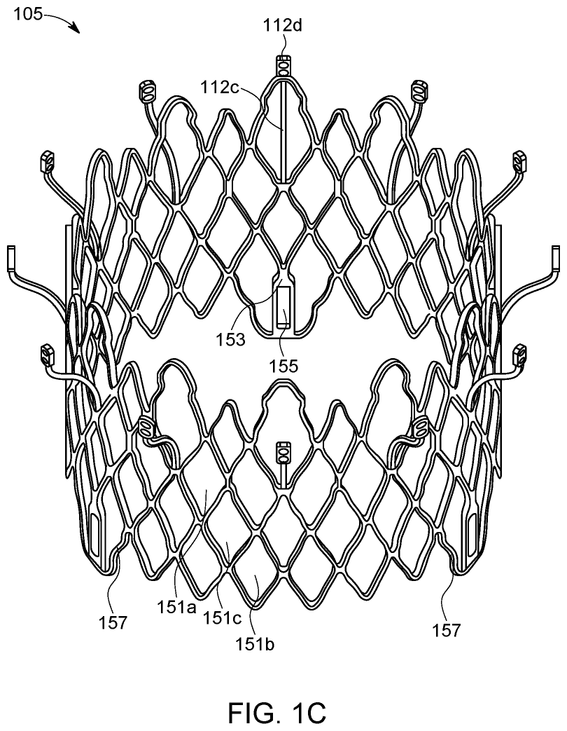

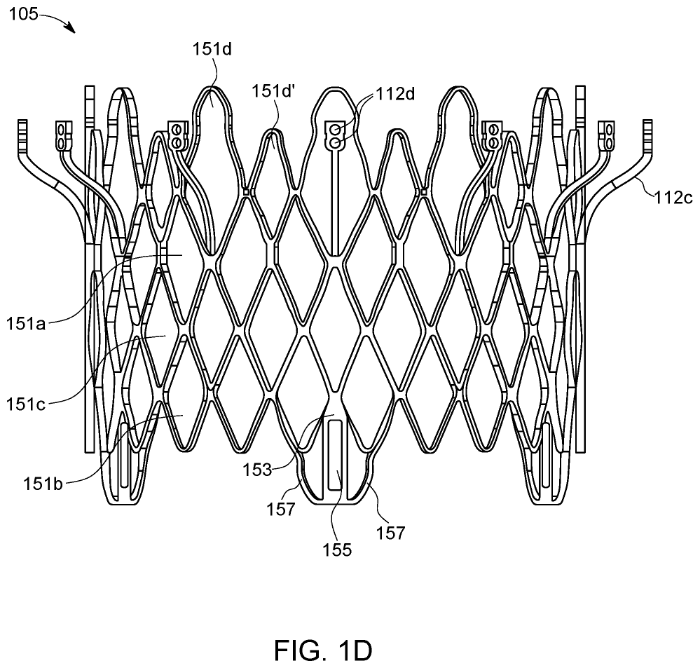

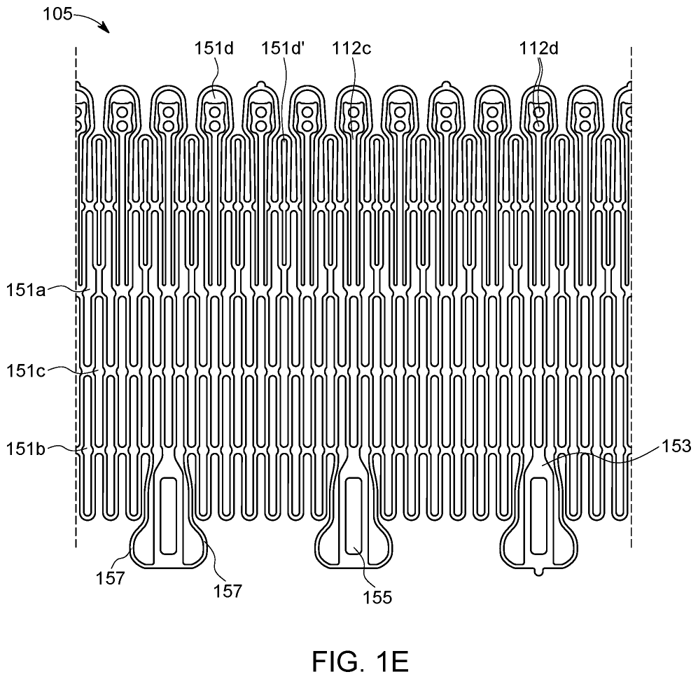

FIGS. 1A-B illustrate an outer frame 101 for use with a prosthetic heart valve, while FIGS. 1C-E illustrate an inner frame 105 for use with a prosthetic heart valve. The prosthetic heart valve that incorporates outer frame 101 and inner frame 105 may be particularly suited for replacement of a native mitral or tricuspid valve. The outer frame 101 may be primarily for anchoring the prosthetic heart valve within the native heart valve annulus, while the inner frame 105 may be primarily for holding the prosthetic valve assembly (e.g., a plurality of prosthetic leaflets, with or without additional sealing skirt(s)) in the desired position and orientation.

Outer frame 101 is illustrated in FIG. 1A isolated from other components of the prosthetic heart valve. In FIG. 1A, the outer frame 101 is illustrated in an expanded condition. In FIG. 1B, the outer frame 101 is illustrated in an unexpanded condition, as if cut longitudinally and laid flat on a table. As used herein, the term “unexpanded” refers to the state of the frame prior to being shape-set (e.g. immediately after it is cut from a tube of nitinol). After being formed, the frame may be shape-set to the expanded condition, and may also have a collapsed condition in which the frame is collapsed to a smaller size from its expanded condition. The shape of the frame may be similar, but not necessarily identical, in the unexpanded and collapsed conditions. As shown in FIG. 1A, outer frame 101 may include an atrial portion, flare, flange, or anchor 102, a ventricular portion, flare, flange, or anchor 104, and a central waist portion 103 coupling the atrial portion to the ventricular portion. The central portion 103 may be between atrial portion 102 and ventricular portion 104. Atrial portion 102 may be configured and adapted to be disposed on an atrial side of a native valve annulus, and may flare radially outwardly from the central portion 103 so that the central portion 103 is in the form of a central waist. Ventricular portion 104 may be configured and adapted to be disposed on a ventricle side of the native valve annulus, and may also flare radially outwardly from the central portion 103. The central portion 103 may be configured to be situated in the valve orifice, for example in contact with the native valve annulus. In use, the atrial portion 102 and ventricle portion 104 effectively overlie the native valve annulus on the atrial and ventricular sides thereof, respectively, helping to hold the prosthetic heart valve in place.

The atrial portion 102, central waist 103, and ventricular portion 104 may each be formed by a plurality of generally diamond-shaped cells, although other suitable cell shapes, such as triangular, quadrilateral, or polygonal may be appropriate. In some examples, the outer frame 101 may be formed as a braided mesh, as a portion of a unitary frame, or a combination thereof. According to one example, the outer frame 101 may be laser cut from a tube of nitinol and heat treated to a desired shape so that the outer frame 101 is collapsible for delivery, and re-expandable to the set-shape during deployment. The outer frame 101 may be formed from other materials, including other super-elastic and/or shape-memory metals or metal alloys other than nitinol, or a plastically expandable material such as cobalt chromium. The various portions of the outer frame 101 may be heat treated into a suitable shape to conform to the native anatomy of the valve annulus to help provide a seal and/or anchoring between the outer frame 101 and the native valve annulus. The outer frame 101 may be partially or entirely covered by a cuff or skirt, on the luminal and/or abluminal surface of the outer frame 101, although it may be preferable to keep at least a portion of the ventricular anchor 104 uncovered by a cuff or skirt to help maximize flow to the left or right ventricular outflow tract. If included, the cuff or skirt may be formed of any suitable material, including biomaterials such as bovine pericardium, biocompatible polymers such as ultra-high molecular weight polyethylene (“UHMWPE”), woven polyethylene terephthalate (“PET”) or expanded polytetrafluoroethylene (“ePTFE”), or combinations thereof. The atrial portion 102 may include features for connecting the atrial portion to a delivery system. For example, the atrial portion 102 may include pins or tabs 122 around which sutures (or suture loops) of the delivery system may wrap, so that while the suture loops are wrapped around the pins or tabs 122, the outer frame 101 maintains a connection to the delivery device.

Referring to FIG. 1A, outer frame 101 may include a plurality of rows of diamond-shaped cells. For example, the outer frame 101 may include twenty-four diamond-shaped cells in the ventricular-most rows of cells 111c, which may be referred to as the “ventricular petals” as they are intended for positioning on a ventricular side of the native mitral valve annulus. In the particular illustrated example, outer frame 101 includes only a single row of ventricular cells 111c, although more than one row of ventricular cells may be provided in other embodiments. Further, outer frame 101 may include three rows of diamond-shaped cells at the central waist portion 103 of the outer frame 101, although more or fewer rows may be suitable in other examples.

The atrial or inflow end of outer frame 101 may include a first row of atrial cells that include a first type of atrial cell 111a that alternates with a second type of atrial cell 111a ′. The first type of atrial cells 111a and the second type of atrial cells 111a′ may both be generally diamond-shaped, with the first type of atrial cells 111a being slightly wider than the second type of atrial cells 111a′ in the circumferential direction. The first type of atrial cell 111a may also extend slightly farther in the inflow direction than the second type of atrial cell 111a′. Outer frame 101 may include twelve of each type of atrial cell 111a, 111a′ for a total of twenty-four cells. By having a relatively large number of atrial cells in outer frame 101, the atrial portion 102 of outer frame 101 may be exposed to lower forces during loading, deployment, and while in use, which may reduce strain experienced by the atrial portion of the outer frame 101 and thus increase durability of the outer frame 101 compared to a similarly sized frame with fewer cells.

Still referring to FIG. 1A, it should be understood that pins or tabs 122 may be included with outer frame 101. If twelve pins or tabs 122 are provided, they may be provided in the wider first type of atrial cell 111a, and omitted from the narrower second type of atrial cell 111a′. As a result, when the outer frame 101 is expanded, there is a relatively large amount of clearance around the pin or tab 122. With this configuration, after the outer frame 101 is fully expanded, it may be relatively easy to withdraw any suture loops that are positioned around the pin or tab 122, for example by advancing the suture loops forward and then beyond the free end of the pin or tab. When the outer frame 101 is in the collapsed condition, however, the clearance around the pin or tab 122 may significantly reduce, so that the suture loop is less likely to unintentionally detach, since the available space for the suture loops to move is reduced.

Outer frame 101 may also include a second row of atrial cells 111b. The second row of atrial cells 111b may include a bottom V-shaped portion that shares struts with cells 111e and 111f (described below), and a top portion that shares struts with an adjacent pair of the first type of atrial cell 111a. The bottom half of the second type of atrial cell 111a′ may be thought of as protruding into the second row of atrial cells 111b, interrupting what would otherwise be a large diamond shape of the second row of atrial cells 111b. As is described in greater detail below, a coupling arm 112a may extend from the bottom apex of each atrial cell 111b and toward the atrial or inflow end.

A first row of center cells 111e may be positioned adjacent the atrial end 102 of the outer frame 101, each cell 111e being positioned between a pair of adjacent atrial cells 111b. Each center cell 111e may be substantially diamond-shaped, but it should be understood that adjacent center cells 111e do not directly touch one another. The first row of center cells 111e may include twelve center cells 111e. A second row of center cells 111f may be positioned at a longitudinal center of the outer frame 101, each center cell 111f being positioned between an atrial cell 111b and center cell 111e. In the illustrated embodiment, center cells 111f in the second row may be diamond-shaped, with the second row including twenty-four center cells 111f. Finally, a third row of center cells 111g may be positioned between the second row of center cells 111f and the row of ventricular cells 111c. The third row of center cells 111g may include twenty-four cells and they may each be substantially diamond-shaped.

Outer frame 101 may include a plurality of ventricular tines 108 that function to frictionally engage tissue on the ventricular side of the native valve annulus to help enhance anchoring. In the illustrated embodiment, each tine 108 extends from an apex of each cell 111c in the ventricular row of cells (the apex being positioned opposite the outflow-most portion of each cell 111c). Thus, outer frame 101 may include twenty-four ventricular tines 108, with each ventricular tine being equidistantly spaced from circumferentially adjacent ventricular tines. However, it should be understood that more or fewer (including zero) ventricular tines 108 may be provided, and the positioning may be different than shown in the particular embodiment shown in FIG. 1A. For example, a fewer number of tines may be evenly spaced. In this regard, every other tine in FIG. 1A may be omitted to allow for such even spacing between adjacent tines. In another example, a number of tines may be removed to relative to anatomical alignment of the native annulus. For example, tines may be omitted on the side of anchor 104 nearest to the aortic valve at the left ventricular outflow tract (LVOT). In other words, in some examples, tines may be omitted from the side of the anchor 104 that will face anteriorly (e.g., to engage the A1, A2, and/or A3 anterior leaflets of the mitral valve) up implantation into the native mitral valve annulus, since the anterior leaflet of the mitral valve is generally positioned adjacent to the aortic valve. This targeted omission of tines in some examples may reduce the occurrence of engagement with the aortic valve, either native or replacement.

Outer frame 101 may include a plurality of coupling arms 112a. Each coupling arm 112a may be a strut that is coupled to a bottom or outflow apex of each atrial cell 111b in the second row, with each strut extending toward the inflow end of the outer frame 101 to a free end of the coupling arm 112a. The free end of each coupling arm 112a may include a pair of vertically spaced apertures 112b for coupling to the inner frame 105, as described in greater detail below. However, it should be understood that fewer or more than two apertures 112bmay be provided in other embodiments, and the relative positioning of apertures 112b may be other than the vertical orientation shown. In the collapsed condition (similar to the unexpanded condition shown in FIG. 1B), each coupling arm 112a is substantially surrounded by an atrial cell 111b in the second row. In the expanded condition, best shown in FIG. 1A, the coupling arms 112a may extend radially inwardly and have a contour so that the free end extends substantially parallel to the center longitudinal axis of the outer frame 101. In the illustrated embodiment, a total of twelve coupling arms 112a is provided along the row of twenty-four center-most cells 111f.

FIGS. 1C-D are perspective and side views, respectively, of an inner frame 105 of a prosthetic heart valve, which may be configured for use with the outer frame 101. FIG. 1E is a flattened view of inner frame 105 as if cut longitudinally and laid out flat on a table in an unexpanded condition. In an assembled prosthetic heart valve, the inner frame 105 may be positioned radially within the outer frame 101 when the inner and outer frames are assembled together.

Inner frame 105 may be primarily intended to support prosthetic leaflets, and may be designed to foreshorten axially upon radial expansion. For example, inner frame 105 may include five rows of cells, most or all of which are generally diamond-shaped. In particular, inner frame 105 may include first, second, and third rows of diamond-shaped cells 151a-151c. In the illustrated embodiment, cells 151b are the outflow-most row of cells, with cells 151c being adjacent cells 151b, and cells 151a being adjacent cells 151c. The three rows of cells 151a-151c may each include twenty-four cells, although some of the cells in the second row 151b may be formed partially with webbed commissures, described below. The third row of cells 151c may be positioned between the first row of cells 151a and the second row of cells 151b.

Inner frame 105 may also act as a stiffening member for the outer frame 101. In general, the outer frame 101, when not engaged with the inner frame 105, exhibits a degree of flexibility, especially to a flat plate crush-style load. Incorporation of the inner frame 105 and engagement of the inner frame 105 with the outer frame 101 can increase the overall stiffness of the inner frame 101 and outer frame 105 assembly as compared to the outer frame 105 alone.

Inner frame 105 may include three generally rectangular-shaped commissure windows 155 equidistantly spaced around the circumference of the inner frame, with each commissure window adapted to provide a location for coupling two adjacent prosthetic leaflets to an axial strut 153. However, more or fewer commissure windows 155 may be provided depending on how many prosthetic leaflets will be coupled to the inner frame 105. As illustrated in FIGS. 1C-E, the commissure windows 155 may be formed in axial struts 153 extending from a location where two adjacent cells 151b in the same row meet. In other words, each axial strut 153 extends from an outflow apex of a cell 151c. With this configuration, the webbed commissures of inner frame 105 only extend beyond the row of cells 151b in the outflow direction a length of about one-half the axial length of a cell 151b. The webbed commissures of inner frame 105 thus may only extend minimally into the ventricle upon implantation, which may reduce the likelihood (or amount of) obstruction of the LVOT (if used as a prosthetic mitral valve) or RVOT (if used as a prosthetic tricuspid valve). Additional support struts 157 may connect the axial struts 153 to the cells 151b. In particular, two support struts 157 may connect the axial strut 153 to the inner frame 105 at side apices of cells 151b. The positioning of the webbed commissures of inner frame 105 may allow the free edge of the prosthetic leaflets to be fully exposed to blood flow without the inner frame 105 blocking the flow, which may provide improved leaflet closing dynamics (e.g. faster leaflet coaptation) and may reduce the likelihood or possibility of leaflet wear due to contact with the inner frame 105. Also, the webbed commissures of inner frame 105 may be structurally stable at least in part due to the support struts 157 that create the “web” of the webbed commissure.

FIG. 1F is a side view of a portion of an inner frame of a prosthetic heart valve, which may be configured for use with the outer frame FIGS. 1A-B. While FIGS. 1C-E depict generally rectangular-shaped commissure windows 155, other shaped windows, including partially open windows, may be implemented.

For example, FIG. 1F depicts a partially-open commissure window 155a at least partially defined by axial struts 153a. As shown in FIG. 1F, the partially-open commissure window 155a can be defined on two sides (in one example, opposing sides) by axial struts 153aand on another side by the outflow apex 153c of the adjacent cell in row 151c. The combination of axial struts 153a and outflow apex 153c can be generally U-shaped (e.g., with one side of a rectangle being omitted), thereby defining a pair of freely extending tips 153b with an opening therebetween. In one example, the omitted side of the rectangle is on the outflow side of the rectangle and the opening faces the outflow side of the inner frame. Advantageously, the partially-open commissure window 155a can allow for prosthetic leaflets to be coupled to the inner frame 105 via partially-open commissure window 155a by sliding the prosthetic leaflet from the outflow direction toward the inflow direction. In other words, in some examples, instead of passing portions of prosthetic leaflets through a closed commissure window (such as commissure window 155) from the luminal to abluminal surface of the inner frame 105, the prosthetic leaflet commissure (or a portion thereof) may be axially slid into the commissure window 155a via the open end between tips 153b.

As also shown, the supporting struts 157 depicted in FIGS. 1C-E may be omitted. In this example, the axial struts 153a are connected only to a single outflow apex 153c of the adjacent cell in row 151c and the freely extending tips 153b can be free from connection to any outflow apexes 153d of the adjacent cell or cells in row 151c. This configuration can allow for the axial struts 153a to deflect inwardly, e.g., farther inwardly than in the configuration depicted in FIGS. 1C-E, to allow for the stress otherwise placed on the prosthetic leaflets to be distributed to inner frame 105.

While the example of FIG. 1F depicts both partially-open commissure windows 155a and the omission of supporting struts 157, either or both of these modifications can be incorporated in the example of FIGS. 1C-E to achieve the desired deflection of axial struts.

Referring to FIG. 1E, it may be desirable to allow for the axial struts 153 to deflect radially inwardly during normal operation of the prosthetic heart valve that incorporates inner frame 105. For example, adjacent prosthetic leaflets are coupled to each other and to the inner frame 105 via commissure window 155. Thus, during ventricular systole when the prosthetic leaflets coapt and are subjected to the relatively large pressures of ventricular systole, the force on the prosthetic leaflets is transmitted to their connection to the inner frame 105, including at axial struts 153. If the axial struts 153 with commissure windows 155 were extremely rigid, a relatively high amount of stress would be applied to the prosthetic leaflets where they couple to the axial struts 153. However, by designing the axial struts 153 to be capable of deflecting inwardly, some amount of the stress that would otherwise be placed on the prosthetic leaflets is instead distributed to the inner frame 105. By reducing the stress on the prosthetic leaflets, the prosthetic leaflets may be more durable than if the prosthetic leaflets had to bear a relatively higher amount of the stress during ventricular systole. The webbed commissures of inner frame 105 may be specifically designed so that they are capable of flexing between about 0.5 mm and about 1.5 mm, including about 1.0 mm, radially inwardly (measured at the tips of the axial struts 153) during ventricular systole. Some of the parameters that may be leveraged to fine-tune the amount of deflection that will occur in the axial struts 153 include (i) the wall thickness of the frame (in the direction into the page in the view of FIG. 1E); (ii) the width of the node (in the left-to-right direction in the view of FIG. 1E) where the axial strut 153 connects to the outflow apex of the adjacent cell in row 151c; and (iii) the axial position along axial strut 153 where supporting struts 157 connect to axial strut 153. In one embodiment, to achieve a desired amount of deflection, the width of the node may be larger than the wall thickness of the inner frame 105. For example, the ratio of the width of the node to the wall thickness may be greater than 1 and less than 2, including about 1.3, about 1.4, or about 1.5. It may be preferable, as shown in FIG. 1E, to couple the support struts 157 to the terminal (otherwise free) end of the axial strut 153. This may reduce deflection to help achieved the desired amount of deflection. Further, by connecting the support struts 157 to the outflow-most end of the axial struts 153, the support struts 157 may curve outwardly and back inwardly at positions beyond the ends of the cells in the second row of cells 151b. This curvature may provide for extra length of the support struts 157. This extra length may assist in ensuring that the cells connected to the opposite ends of the support struts 157 are capable of expanding as intended. In other words, when the inner frame 105 transitions to the expanded condition shown in FIGS. 1C-D, the axial struts 153 do not undergo any conformational change, while the adjacent cells do. The extra length of support struts 157 resulting from the above-described contouring provides for cells adjacent to the axial struts 153 that are not restricted from expanding, despite the axial struts themselves remaining unchanged during expansion.

Inner frame 105 may also include a fourth row of cells at the inflow-most (or atrial) end of the frame. The fourth row of cells may include a first type of atrial cell 151d that alternates with a second type of atrial cell 151d′ for a total of twenty-four cells in the atrial-most row. The second type of atrial cell 151d′ may be a diamond-shaped cell coupled to a runner between pairs of cells 151a in the first row. The first type of atrial cell 151d may be generally diamond-shaped, but larger than the second type of atrial cell 151d′. The first type of atrial cell 151d may extend a greater length in the inflow direction than the second type of atrial cell 151d′, and the first type of atrial cell 151d may surround corresponding coupling arms 112c, described in greater detail below, at least when the inner frame is in the unexpanded condition shown in FIG. 1E. Although inner frame 105 is described as having four rows of cells, it may also be appropriate to describe inner frame 105 as including five row of cells if the webbed commissures are counted as a row of cells separate from the second row of cells 151b.

Inner frame 105 may include coupling arms 112c coupled to points where every other pair of adjacent cells 151a in the first row meet, resulting in a total of twelve coupling arms 112c. Coupling arms 112c may include two vertically separated apertures 112d. However, the number and positioning of the coupling arms 112c, as well as the number and positioning of apertures 112d, may be different than the particular example shown. It is preferable that the number and positioning of the coupling arms 112c, as well as the number and positioning of apertures 112d, are complementary to the number and positioning of the coupling arms 112a, as well as the number and positioning of apertures 112b.

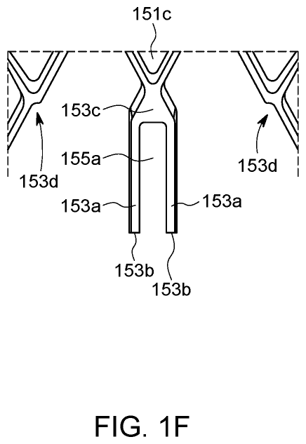

As should be understood from the above description, the outer frame 101 may be attached to the inner frame 105, and additional components such as prosthetic leaflets and fabric cuffs/skirts may be attached to the frame(s) in order to form a self-expanding prosthetic mitral (or tricuspid) valve. In use, a single-size inner frame 105 may be used with different-sized outer frames 101 in order to accommodate different patient anatomies. For example, the inner frame 105 may have a 27 mm diameter, and the outer frame 101 may come in different diameters (at the central waist) of 32 mm, 36 mm, or 40 mm. It should be understood that these are merely exemplary sizes, and the inner frame could be provided in additional sizes, and the outer frame could be provided in more or fewer sizes than listed above. The size of the outer frame may affect the particular geometry of the coupling arm 112a, particularly if only a single size of inner frame 105 is provided. For example, the outer frame 101 illustrated in FIGS. 1A-B may have a diameter of 40 mm at the central waist. The coupling arm 112a, when in the expanded condition, extends upward (or in the atrial direction) from its point of connection to the outer frame 101, and then radially inwardly. As shown in FIG. 2A, when the inner frame 105 is assembled to the outer frame 101 (having a 40 mm diameter), the coupling arms 112a, 112c extend toward each other, curving near their free ends so that the terminal flat plates press against each other in a vertical direction and the two apertures 112b align with the two apertures 112d. The terminal free ends (or flat plates) of the coupling arms 112a, 112c may be coupled via suturing through the corresponding apertures 112b, 112d, or by any other suitable fastener (such as rivets). The geometry of the coupling arms 112a, 112c may result in the arms meeting at a point about mid-way between the outer diameter of the inner frame 105 and the inner diameter of the outer frame 101. Although not shown, a buffer material (such as fabric or tissue) may be provided between the terminal free ends of the coupling arms 112a, 112c, for example to prevent direct metal-to-metal contact. Although fasteners such as rivets may be used to couple any of the inner and outer frames described herein, it may be preferable to use sutures to fasten the coupling arms for ease of manufacturing (e.g. the inner and outer frames may be coupled to one another relatively late in the manufacturing process). In addition, although rivets are a suitable fastening option, rivets may create a potential for galvanic corrosion that may occur when a metal rivet is used to couple metal frames, where the metal of the rivet is dissimilar to the metal of the frames. The use of suture fasteners may eliminate this potential issue.

If outer frame 101 has a diameter smaller than the 40 mm diameter shown in FIGS. 1A-B, the coupling arm of the outer frame may have a slightly different geometry. For example, if outer frame 101 has a 36 mm diameter at the central waist, the coupling arm 112a′ may extend substantially fully vertically from the outer frame, as shown in FIG. 2B. Because the inner frame 105 in FIGS. 2A-B is identical, the different geometry of coupling arm 112a′ of the smaller diameter outer frame 101 may be necessary in view of the smaller diameter of the outer frame 101. This same concept may be applied to a variety of sizes of outer frame 101. For example, FIG. 2C illustrates an even smaller size outer frame 101, which may have a 32 mm diameter at the central waist, while again inner frame 105 remains identical. In order to accommodate this even smaller size outer frame 101, coupling arms 112a′' may extend vertically from their connection point with the outer frame 101, and then radially outwardly, before returning to a vertical direction. Additional diameters of outer frame 101 may be provided with inner frame 105, and the coupling arms 112a of the outer frame may be modified in ways other than those explicitly illustrated to allow for coaxial positioning of the outer frame around the inner frame, with uniform contact between coupling arms 112a and 112c. It should be understood that in FIGS. 2A-2C, only a single coupling arm of the outer frame is illustrated, with the remainder of the outer frame omitted, for clarity of illustration.

Referring back to FIG. 1A, whether outer frame 101 has a relatively large diameter or a relatively small diameter, the coupling arms 112a preferably couple to the outer frame 101 at or near the middle of the central waist portion of the outer frame, for example at or near the smallest diameter portion of the outer frame. Both coupling arms 112a of the outer frame 101 and coupling arms 112c of the inner frame 105 are preferably balanced in stiffness relative to each other so that, as the inner and outer frame assembly collapses or expands, the point where the coupling arms couple remains generally between the inner and outer frames, without significantly moving away from the outer frame toward the inner frame, or significantly moving away from the inner frame toward the outer frame. This may help maintain balance between the inner and outer frames, while avoiding excess strain being placed on coupling arms of the inner frame or the outer frame during collapsing or expansion. The positioning and design of the coupling arms may provide additional benefits as well. During a typical transseptal delivery, the ventricular end of the prosthetic heart valve is deployed from the delivery device first, with the atrial end deploying last. Because the coupling arms 112c of the inner frame 105 are positioned on the atrial side of the inner frame, with the actual point of connection of the coupling arms 112c to coupling arms 112a being near the atrial-most end of the inner frame, the inner frame 105 will tend to be positioned a significant distance within the delivery sheath, even as the ventricular end of the outer frame 101 begins to deploy from the delivery sheath. By maintaining a significant length of the inner frame 105 collapsed within the delivery device while the ventricular end of the outer frame 101 begins to deploy, the prosthetic heart valve may deploy in a more controlled manner.

As explained above, the outer frame 101 and inner frame 105 couple to each other via coupling arms 112a, 112c that are both formed as single struts. Depending on the particular conditions of use, this design may create certain obstacles. Shape-memory material such as nitinol may be subject to failure if enough strain is applied to the material, including large strains over a short time period or smaller cyclical strains over a longer period. For example, if a prosthetic heart valve incorporating outer frame 101 and inner frame 105 were implanted into a native mitral valve, which is subject to high cyclical pressures, every time the left ventricle contracts and the prosthetic leaflets within inner frame 105 close, the coupling arms 112a and/or 112c need to provide a countering force as the pressure within the left ventricle tends to move the inner frame 105 upward toward the left atrium. Thus, stain is placed on the coupling arms 112a and/or 112c each time the left ventricle contracts, dozens of times per minute. High cyclic strains and high sheathing strains can reduce the durability of frame material, and nitinol frame material in particular. For example, cyclical strain could result in a fracture of one or more of the coupling arms 112a and/or 112c. Other designs may reduce the cyclic and sheathing strains. Self-expanding prosthetic heart valves are expected to have other, non-cyclical strains applied to them as well. For example, prior to delivering a prosthetic heart valve, it needs to be collapsed and pulled into a delivery device, and that motion needs to be generally reversed when deploying the prosthetic heart valve into the patient. These sheathing and unsheathing (and potential re-sheathing) actions typically apply a large amount of strain to the outer frame 101 and the inner frame 105. This is another source of potential fracturing of coupling arms 112a and/or 112c. Within a nitinol frame design, it may be important to have redundancy. In other words, in the event that a frame fracture was to occur, the impact of the fracture may be less likely to cascade to other parts of the frame if there is redundancy in the design. As a specific example, while it would generally be undesirable for strut connections between outer frame 101 and outer frame 105 to fracture, it may be particularly undesirable given that each coupling arm 112a and 112c has only a single point of connection to its respective frame. In other words, because there is no redundancy in any one pair of coupling arms 112a and 112c, a fracture in either coupling arm 112a or 112c of a pair of coupling arms could lead to significant disruption of the safe functioning of the prosthetic heart valve. As is described in greater detail below, by modifying the coupling arms 112a, 122c to be multiple-strut (e.g., double strut) connectors, instead of single strut connectors, the likelihood of fracture may be reduced, and even if a fracture does occur, the coupling arms may have redundancy to lessen or eliminate any negative impact of such fracture.

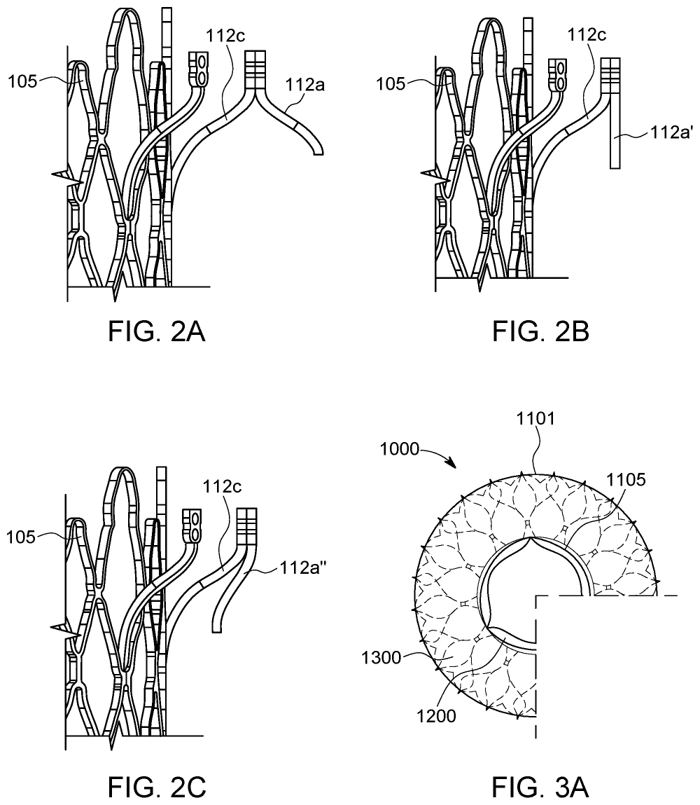

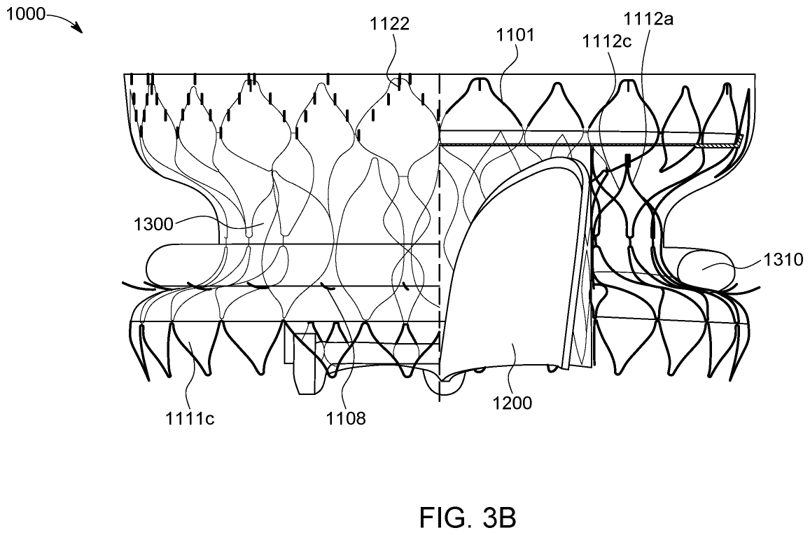

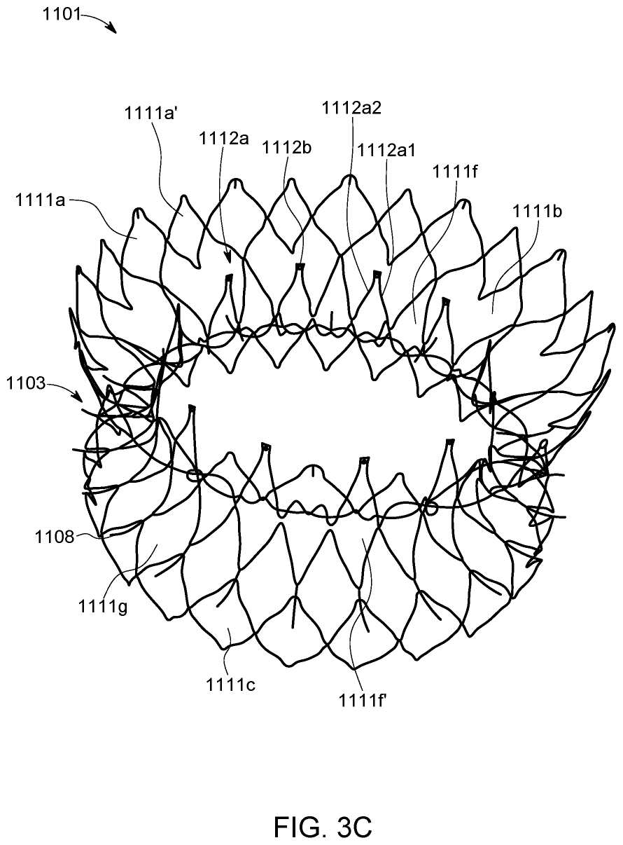

FIG. 3A is a top-down view of a prosthetic heart valve 1000 according to another aspect of the disclosure, with a one-quarter or 90-degree radial slice of the prosthetic heart valve 1000 being removed. FIG. 3B illustrates prosthetic heart valve 1000 from the side with the same slice removed.

Generally, prosthetic heart valve 1000 may be an expandable and collapsible prosthetic heart valve including an outer frame 1101, inner frame 1105, a plurality (e.g., three) of prosthetic leaflets 1200 disposed within the inner frame 1105, and one or more sealing skirts 1300 positioned on interior and/or exterior surfaces of the prosthetic heart valve 1000 to help ensure blood is unable to flow through the prosthetic heart valve 1000 other than via the central lumen defined by prosthetic leaflets 1200 when they are in the open condition shown in FIG.

3A.

Referring now to FIG. 3B, it should be understood that outer frame 1101 may be substantially identical to outer frame 101, and inner frame 1105 may be substantially identical to inner frame 105, with certain exceptions described below. Thus, the features of the outer frames 101, 1101 and inner frames 105, 1105 that are the same are not described again here in detail for brevity. Three prosthetic leaflets 1200 may be coupled to inner frame 1105, for example via commissure windows 1155. The prosthetic leaflets 1200 may be formed of tissue (e.g., bovine or porcine pericardium) or synthetic materials (e.g., PET, PTFE, UHMWPE, etc.). The skirt 1300 may be provided at least on an outer surface of the outer frame 1101, and crossing an area (e.g. on the atrial side of the prosthetic heart valve 1000) between the outer frame 1101 and the inner frame 1105 to help seal against blood flowing between the frames or around the outside of the outer frame 1101. In the illustrated example, the skirt 1300 has cutouts that align with pins 1122 and tines 1108 of the outer frame 1101. Further, in the illustrated embodiment, the skirt 1300 does not cover the outflow-half of ventricular cells 1111c to help prevent obstruction of the left (or right) ventricular outflow tract. Still further, in the illustrated embodiment, the skirt 1300 includes a bumped portion or protrusion 1310. The protrusion 1310 may be generally tubular and extend around the circumference of the outer frame 1101 between the central waist 1103 of the outer frame 1101 and the position of the tines 1108. This protrusion 1310 may help create better sealing contact with the native valve annulus to further prevent blood from leaking around the outside of the prosthetic heart valve 1000. The skirt 1300 may be coupled to the outer frame 1101 and/or inner frame 1105 via any suitable mechanism, including for example via sutures that follow struts of the outer frame 1101 and/or inner frame 1105.

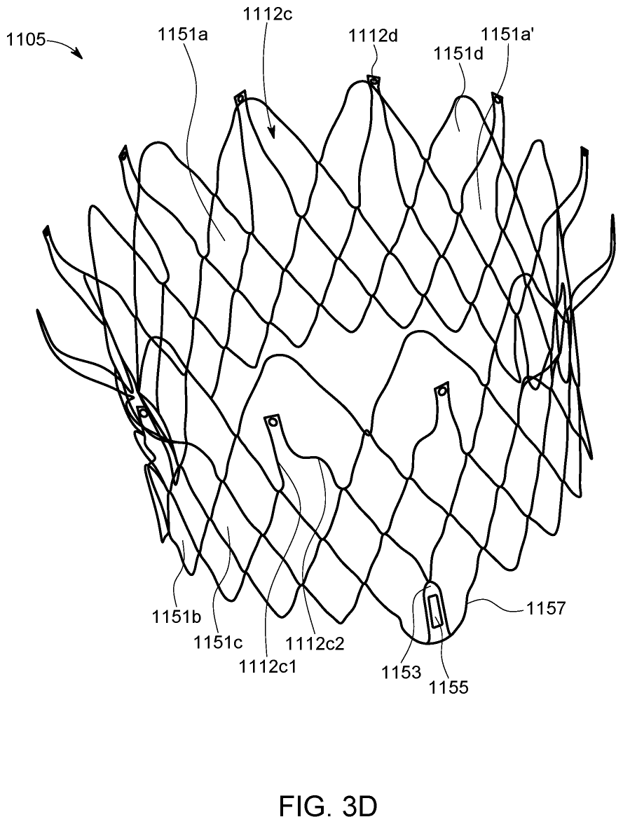

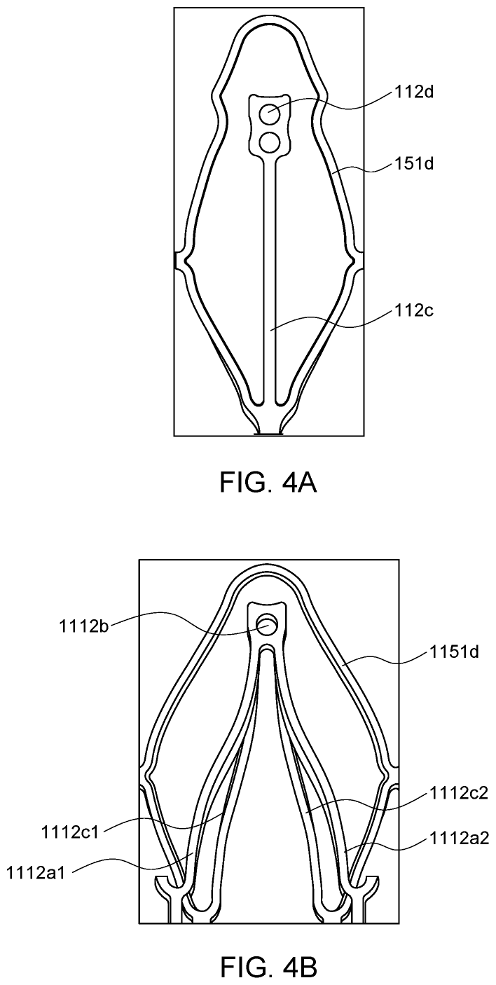

As shown in the cutaway portion of the view of FIG. 3B, the inner frame 1105 may include a plurality of coupling arms 1112c that are each coupled to a corresponding coupling arm 1112a of the outer frame 1101, for example via sutures, rivets, or other fasteners. The main difference between inner frame 105 and 1105, and outer frame 101 and 1101, is that the coupling arms 1112a, 1112c are each formed having a double strut configuration. The single strut configuration of inner frame 105 and outer frame 101 is shown in FIG. 4A next to the double strut configuration of inner frame 1105 and outer frame 1101 in FIG. 4B for a direct comparison.

Outer frame 1101 is illustrated in FIG. 3C, in an expanded condition, isolated from other components of the prosthetic heart valve 1000. As shown in FIG. 3C, the outer frame 1101 may include a plurality of diamond-shaped cells in the ventricular-most row of cells 1111c which may be similar or identical to cells 111c of outer frame 101 and are thus not described in detail again here. Next to the ventricular-most row of cells 1111c, the outer frame 1101 may include a ventricular-side row of center cells 1111g that may be substantially similar or identical to cells 111g of outer frame 1101. A plurality of tines 1108 may extend from the point where each pair of adjacent cells 1111g meet, in a substantially similar or identical manner to tines 108. The atrial end of the outer frame 1101 may include a plurality of first atrial cells 1111a alternating with a plurality of second atrial cells 1111a′, which may be similar or identical to cells 111a and 111a′, respectively, of outer frame 101.

As noted above, the main difference between outer frame 101 and outer frame 1101 is that outer frame 1101 has a single strut coupling arm 112a, while outer frame 1101 has a double strut coupling arm 1112a. However, it should be understood that in other embodiments, the coupling arm 1112a may include more than two struts. As is explained below, while a double strut configuration may be desirable, more than two struts may also provide benefits over the single strut embodiment. In order to accommodate the double strut coupling arm 1112a, there are certain changes to the cellular design of outer frame 1101 compared to outer frame 101. For example, whereas outer frame 101 has a generally center row 111f of substantially uniform diamond-shaped cells, outer frame 1101 has a center row of cells at the central waist 1103 that includes a first type of center cell 1111f and a second type of center cell 1111f′. The first type of center cell 1111f is a generally diamond-shaped cell with an outflow apex that joins to the inflow apex of a corresponding ventricular cell 1111c. The first type of center cell 1111f has an inflow apex that joins to the outflow apex of a corresponding one of the first type of atrial cells 1111a (which may include a pin 1122). Each first type of center cell 1111f is positioned between a circumferentially adjacent pair of second type of center cell 1111f′, so that the first and second types of center cells alternate with each other around the circumference of the outer frame 1101. The second type of center cell 1111f′ is also generally diamond-shaped with an outflow apex that joins to the inflow apex of a corresponding ventricular cell 1111c. However, the inflow apex of the second type of center cell 1111f′ is not directly coupled to the outer frame 1101, but rather defines a free end that is positioned radially inwardly of the atrial and ventricular flares of the outer frame 1101. It is the free inflow end of the second type of center cell 1111f′ that defines the double strut coupling arm 1112a. In other words, the inflow end of the second type of center cell 1111f′ includes a first strut 1112a1 that joins a second strut 1112a2 at an inflow apex of the cell, with one or more apertures 1112b formed at the point of joinder.

Still referring to FIG. 3C, the outer frame includes a second row of atrial cells 1111b that are generally similar to the second row of atrial cells 111b of outer frame 101. These cells 1111b are similar in the sense that the coupling arm 1112a nests within cell 1111b when the outer frame 1101 is in the collapsed condition, and in the sense that the cell 1111b has the shape of an enlarged diamond that is interrupted by the coupling arm 1112a and the second type of atrial cell 1111a′ protruding into the second atrial cell 1111b. However, it should be understood that the change in design to outer frame 1101 effectively eliminated the need for the cells 111e of outer frame 101, which is because the atrial side of the outer frame 1101 has a slight rotational shift (of about 7.5 degrees or about a half-cell width). This slightly different rotational orientation helps to allow for the first type of center cell 1111f to be directly coupled to the first type of atrial cell 1111a, and for the coupling arms 1112a to include two struts 1112a1, 1112a2 that are able to nest within the second atrial cell 1112b in the collapsed condition. In other words, the ventricular half of outer frame 101 is similar to the ventricular half of outer frame 1101, and the atrial half of outer frame 101 is generally similar to the atrial half of outer frame 1101, and the outer profile of outer frame 101 is highly similar to the outer profile of outer frame 1101, although the relative rotational positioning between the atrial and ventricular half of outer frame 101 is shifted compared to the relative rotational positioning between the atrial and ventricular half of outer frame 1101.

Inner frame 1105 is illustrated in FIG. 3D, in an expanded condition, isolated from other components of the prosthetic heart valve 1000. As shown in FIG. 3D, the inner frame 1105 may include a plurality of diamond-shaped cells in the ventricular-most rows of cells 1151b which may be similar or identical to cells 151b of inner frame 105. Next to the ventricular-most row of cells 1151b, in the inflow direction, the inner frame 1105 may include a row of diamond-shaped cells 1151c, which may be similar or identical to cells 151c of inner frame 105. Outer frame 1105 may include three commissure windows 1155, similar to outer frame 105, although more or fewer commissure windows 1155 may be provided if more or fewer than three prosthetic leaflets 1200 are provided. The commissure windows 1155 may be formed in axial struts 1153 that are generally similar to axial struts 153. However, in the embodiment of FIG. 3D, the axial struts 1153 have an inflow end coupled to the outflow apex of one of the cells 1151c, which is slightly different than the attachment configuration of inner frame 105 in which the axial struts 153 have an inflow end coupled to a point where two cells in the same row meet. Two support struts 1157 may extend from opposite sides of the outflow end of the axial strut 1153, with each support strut 1157 having a second end coupled to the outflow apex of a cell 1151c in the second row, on either side of the cell 1151c to which the axial strut 1153 is connected.

The main difference between inner frame 1105 and inner frame 105 is the inclusion of a double strut coupling arm 1112c, instead of the single strut coupling arm 112cof inner frame 105. However, it should be understood that in other embodiments, the coupling arm 1112c may include more than two struts. As is explained below, while a double strut configuration may be desirable, more than two struts may also provide benefits over the single strut embodiment. Other changes on the atrial side of the inner frame 1105 are also made, generally to accommodate the inclusion of the double strut coupling arm 1112c. For example, while outer frame 105 includes a complete first row of substantially uniform cells 151a, outer frame 1105 instead includes a first type of cell 1151a that alternates with a second type of cell 1151a′ around the circumference of the outer frame 1105. The first type of cell 1151a is generally diamond-shaped with an outflow apex that joins to an inflow apex of a ventricular cell 1151b, and an inflow apex that joins to a position where two circumferentially adjacent atrial-most cells 1151d meet. The second type of cell 1151a′ is also generally diamond-shaped with an outflow apex that joins to an inflow apex of a ventricular cell 1151b. However, the inflow apex of the second type of cell 1151a′ is a free end that is not directly coupled to the inner frame 1105, the inflow apex forming the coupling arm 1112c. In the particular illustrated example, each coupling arm 1112c includes a first strut 1112c1 joined to an outflow end of a cell 1151c in the second row, and a second strut 1112c2 joined to the outflow end of a circumferentially adjacent cell 1151c. The two struts 1112c1, 1112c2 extend toward each other in the inflow direction and meet at an apex, with one or more apertures 1112d being formed in the apex. Preferably, the coupling arms 1112c are shape set to extend radially outwardly from the remainder of the inner frame 1105. Compared to frame 105, atrial cells 1151d are larger than atrial cells 151d in order to accommodate the larger (double strut vs. single strut) coupling arms 1112c. Thus, inner frame 1105 does not include smaller diamond-shaped cells (like cells 151d′ of inner frame 105) between adjacent atrial cells 1151d. Compared to the atrial portion of inner frame 105, the atrial portion of inner frame 1105 may have a slight rotational shift (of about 7.5 degrees or about a half-cell width) relative to the ventricular portion of the inner frame 1105. This can be seen, for example, by comparing FIG. 1D with FIG. 3D, where the commissure window 155 is axially aligned with the center of coupling arm 112c, versus the commissure window 1155 not being axially aligned with the center of coupling arm 1112c. However, the ventricular portions of inner frames 105 and 1105 may be identical or substantially identical (but for slight differences in the configurations of commissure windows 155 and 1155), with a similar overall height and an identical diameter.

FIGS. 4A-B show enlarged isolated views of a single strut coupling arm (e.g. coupling arm 112c) and double strut coupling arms (e.g. coupling arms 1112a and 1112c), respectively. As described above, a significant amount of strain may be applied to one or both coupling arms, particularly during sheathing and unsheathing of the prosthetic heart valve, as well as during ventricular systole when the prosthetic leaflets 1200 are closed. Because coupling arm 112c is formed as a single strut and has a single point of attachment to the inner frame 105, the coupling arm 112c may be more prone to side-to-side bending upon application of force. Coupling arms 1112a, 1112c, on the other hand, are formed as two connected struts that form part of a closed cell. Compared to coupling arm 112c, coupling arms 1112a, 1112cmay have significantly better resistance to such side-to-side bending. Similarly, coupling arm 112c may be at risk of twisting (e.g., rotating about the longitudinal axis of the single strut arm) upon application of any twisting forces to the coupling arm 112c. Compared to coupling arm 112c, coupling arms 1112a, 1112c may have significantly better resistance to such twisting, since the two struts (1112a1-2, 1112c1-2) are each coupled to portions of a cell, as well as to each other at an apex. Bending and/or twisting of a single strut coupling arm, which may occur during sheathing or unsheathing of the prosthetic heart valve into or from a delivery device as well as during the regular cyclical motion of the heart, may increase the likelihood of fracture of the single strut coupling arm. Because the double strut coupling arms are more resistant to such movement, they may be less prone to fracturing. And as described above, even if one of the two struts (1112a1-2, 1112c1-2) does fracture, the other strut of the coupling arm remains attached to the frame and provides redundancy not found in the single-strut coupling arm.

There may still be further benefits of using a double strut connecting arm compared to a single strut connecting arm. Still referring to FIGS. 4A-B, the node to which coupling arm 112c is attached includes three individual struts extending from the top side of the node, including the single strut of the coupling arm 112c, as well as the two struts of cell 151d that join with the node to which coupling arm 112c is connected. The double strut coupling arms 1112a, 1112c, on the other hand, only have two struts extending from the same side of a single node. The “trident” configuration shown in FIG. 4A may result in a small radius of curvature at the nodes, compared to the two-strut embodiment shown in FIG. 4B. The larger radiuses of FIG. 4B may allow for a reduction in sheathing strains compared to the smaller radiuses of FIG. 4A.

Referring briefly back to FIG. 3D, the struts 1112c1, 1112c2 of the coupling arm 1112c of the inner frame 1105 may both be connected to the inner frame 1105 at a point that is generally in the middle of the inner frame 1105 in the axial direction between the inflow and outflow ends. This positioning may allow for more space to be available to draw the coupling arm 1112c radially outwardly to meet the corresponding coupling arm 1112a of the outer frame 1101, without needing to increase the height of the inner frame 1105. This feature may help to reduce the overall height (in the inflow-to-outflow direction) of the prosthetic heart valve 1000, while increasing the size (e.g. diameter) of the outer frame 1101 that is capable of connecting to the inner frame 1105.

Compared to the inner frame 105 and outer frame 101 of FIGS. 1A-E, the reformatting of the cell design described in connection with the frames of FIGS. 3A-4B may allow for more metal to be allocated to the coupling arms. Although both embodiments are shown with twelve connections between the inner and outer frames, each connection of the latter embodiment has a total of four arms, instead of the two arms per connection of the former embodiment. This may lead to approximately double the strut width allocated to the connections in the latter embodiments compared to the formed embodiments. In general, more strut width corresponds to greater resistance to bending and/or deflection. For example, if connected arms were cut midway along their length, the total cross-sectional area of the double-arm connection is approximately twice that of the single-arm connection. The bending stiffness of a beam is generally proportional to its cross-sectional area, resulting in greater resistance to bending and/or deflection.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve comprising:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end;
a collapsible and expandable inner frame positioned radially inward of the outer frame, the inner frame including a plurality of inner coupling arms having a first end coupled to the inner frame and a second free end; and
a prosthetic valve assembly coupled to and disposed within the inner frame;
wherein the first end of each outer coupling arm includes at least two struts extending radially inwardly from a remainder of the outer frame, the first end of each inner coupling arm includes at least two struts extending radially outwardly from a remainder of the inner frame, and the second free ends of the outer coupling arms are coupled to the second free ends of the inner coupling arms to couple the outer frame to the inner frame.

2. The prosthetic heart valve of claim 1, wherein the first end of each outer coupling arm includes exactly two struts, and the first end of each inner coupling arm includes exactly two struts.

3. The prosthetic heart valve of claim 2, wherein the first ends of the outer coupling arms are coupled to the outer frame at a location substantially equidistant between an inflow end of the outer frame and an outflow end of the outer frame.

4. The prosthetic heart valve of claim 2, wherein the first ends of the inner coupling arms are coupled to the inner frame at a location substantially equidistant between an inflow end of the inner frame and an outflow end of the inner frame.

5. The prosthetic heart valve of claim 2, wherein the outer coupling arms are integral with the outer frame, and the inner coupling arms are integral with the inner frame.

6. The prosthetic heart valve of claim 2, wherein the second free ends of the outer coupling arms are coupled to the second free ends of the inner coupling arms via mechanical fasteners.

7. The prosthetic heart valve of claim 6, wherein the mechanical fasteners are sutures.

8. The prosthetic heart valve of claim 3, wherein, in an expanded condition of the outer frame, the outer coupling arms are contoured so that the second free ends of the outer coupling arms are substantially parallel to a central longitudinal axis of the outer frame, the central longitudinal axis extending from an inflow end of the outer frame to an outflow end of the outer frame.

9. The prosthetic heart valve of claim 2, wherein, in an expanded condition of the inner frame, the inner coupling arms are contoured so that the second free ends of the inner coupling arms are substantially parallel to a central longitudinal axis of the inner frame, the central longitudinal axis extending from an inflow end of the inner frame to an outflow end of the inner frame.

10. The prosthetic heart valve of claim 2, wherein the inner frame includes a plurality of rows of substantially diamond-shaped cells, including a first row at an inflow end of the inner frame.

11. The prosthetic heart valve of claim 10, wherein in a collapsed condition of the inner frame, each inner coupling arm is nested within one of the cells in the first row of cells at the inflow end of the inner frame.

12. The prosthetic heart valve of claim 2, wherein the inner frame includes a plurality of axial struts, each axial strut extending from an outflow apex of a corresponding diamond-shaped cell within a row of diamond-shaped cells in a direction away from an inflow end of the inner frame, the axial struts defining commissure windows, the prosthetic leaflets of the prosthetic valve assembly being coupled to the axial struts via the commissure windows.

13. The prosthetic heart valve of claim 12, wherein each axial strut is coupled to two cells within the row of diamond-shaped cells via two support struts that each extends between the axial strut and a corresponding one of the two cells.

14. The prosthetic heart valve of claim 13, wherein each support strut has a first end coupled to a terminal end outflow end of the axial strut.

15. The prosthetic heart valve of claim 2, wherein the two struts of the outer coupling arm converge to the second free end of the outer coupling arm, an aperture being formed in the second free end of the outer coupling arm.

16. The prosthetic heart valve of claim 15, wherein the two struts of the inner coupling arm converge to the second free end of the inner coupling arm, an aperture being formed in the second free end of the inner coupling arm.

17. The prosthetic heart valve of claim 16, wherein a suture extends through the aperture in the second free end of the outer coupling arm and through the aperture in the second free end of the inner coupling arm to couple the outer coupling arm to the inner coupling arm.

18. The prosthetic heart valve of claim 2, wherein the inner frame and the outer frame are each formed from a nickel-titanium alloy.

19. The prosthetic heart valve of claim 2, wherein, in an expanded condition of the prosthetic heart valve, the second free ends of the outer coupling arms meet the second free ends of the inner coupling arms at a location that is substantially equidistance between the inner frame and the outer frame.

20. The prosthetic heart valve of claim 19, wherein each inner coupling arm has two separate points of connection to the inner frame so that the inner coupling arm is resistant to side-to-side bending, and each outer coupling arm has two separate points of connection to the outer frame so that the outer coupling arm is resistant to side-to-side bending.

21. The prosthetic heart valve of claim 19, wherein each inner coupling arm has two separate points of connection to the inner frame so that the inner coupling arm is resistant to twisting upon application of torque, and each outer coupling arm has two separate points of connection to the outer frame so that the outer coupling arm is resistant to twisting upon application of torque.

22. The prosthetic heart valve of claim 19, wherein each inner coupling arm has two separate points of connection to the inner frame so that the inner frame is resistant to axial travel relative to the outer frame.

23. The prosthetic heart valve of claim 2, wherein the inner frame includes a plurality of axial struts, each axial strut extending from a single outflow apex of a corresponding diamond-shaped cell within a row of diamond-shaped cells in a direction away from an inflow end of the inner frame, the axial struts defining partially-open commissure windows each defining a pair of freely extending tips, the prosthetic leaflets of the prosthetic valve assembly being coupled to the axial struts via the commissure windows.
